(19) United States
(12) Patent Application Publication
(10) Pub. No.: US 2006/0147400 A1
(43) Pub. Date: Jul. 6, 2006

(54) Composition, especially a cosmetic composition, containing at least one alkyl para-hydroxybenzoate and at least one lipophilic amino acid derivative

(75) Inventor: Bertrand Piot, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'Oreal, Paris (FR)

(21) Appl. No.: 10/537,288

(22) PCT Filed: Nov. 27, 2003
(86) PCT No.: PCT/EP03/14855

§ 371 Date: Jun. 1, 2005

Related U.S. Application Data
(60) Provisional application No. 60/441,324, filed on Jan. 22, 2003.

(30) Foreign Application Priority Data
Dec. 2, 2002 (FR) 02/15154

(51) Int. Cl. A61K 8/368

(52) U.S. Cl. 424/70.1

(57) ABSTRACT

The present invention relates to a cosmetic composition comprising, in a physiologically acceptable medium, at least one alkyl para-hydroxybenzoate and at least one lipophilic amino acid derivative, to its cosmetic uses, and to the cosmetic treatment process comprising the application of a composition according to the invention to the skin. The invention also relates to a process for dissolving an alkyl para-hydroxybenzoate using a lipophilic amino acid derivative. The invention also relates to the use of at least one lipophilic amino acid derivative to prevent the adsorption of at least one alkyl para-hydroxybenzoate onto solid particles.

The present invention relates to a composition, especially a cosmetic composition, comprising an alkyl para-hydroxybenzoate (or paraben), the alkyl group containing from 1 to 6 carbon atoms, and a lypophilic amino acid derivative, and also to its uses in cosmetics and/or dermatology. The invention also relates to a process for dissolving an alkyl para-hydroxybenzoate, the alkyl group containing from 1 to 6 carbon atoms, with a lipophilic amino acid derivative.

It is known practice to use preserving agents, and especially alkyl-para-hydroxybenzoates, the alkyl group containing from 1 to 6 carbon atoms, in cosmetic and/or dermatological compositions, on account of their antifungal properties. However, the use of these preserving agents poses a problem since they are sparingly soluble, in particular in aqueous medium. Specifically, parabens have a tendency to recrystallize, which is reflected by the formation of insoluble white particles, which render unacceptable the visual aspect and the sensory quality of compositions containing them. Furthermore, their antifungal power is thereby reduced, and they no longer act as preserving agents.

To promote the dissolution of parabens, one solution consists in adding to the composition containing them a primary alcohol such as ethanol, a polyol such as a glycol, or a surfactant. However, the addition of an excessive amount of primary alcohol should be avoided, especially in compositions intended to be applied to the face, on account of the irritant nature of the alcohol. Moreover, the addition of an excessive amount of glycols gives the composition a tacky, sticky nature. Furthermore, it is sought to avoid the use of an excessive amount of surfactants on account of their irritant nature to the skin and the eyes, especially in the case of sensitive individuals. In addition, certain anionic and nonionic surfactants are incompatible with parabens, and inhibit their activity. All these drawbacks are exacerbated when it is necessary to increase the amount of parabens in the compositions.

There is thus still a need to be able to introduce these compounds of low solubility, in sufficient amount, into cosmetic and/or dermatological compositions, without losing cosmetic efficacy.

It thus remains necessary to be able readily to dissolve alkyl para-hydroxybenzoates (or parabens) in a physiologically acceptable medium, which results in a minimum of discomfort when applied to the skin or the scalp. In addition, it is necessary to be able to dissolve a sufficient amount of these compounds for the purpose of cosmetic or dermatological use, without recrystallization of these compounds or loss of stability of the composition containing them.

The Applicant has now discovered that lipophilic amino acid derivatives-make it possible, unexpectedly, to increase the dissolution of these parabens.

One subject of the present invention is thus a composition comprising, in a physiologically acceptable medium, at least one alkyl para-hydroxybenzoate, the alkyl group containing from 1 to 6 carbon atoms, and at least one lipophilic amino acid derivative.

The use of the lipophilic amino acid derivatives according to the invention makes it, possible to dissolve a sufficient amount of alkyl para-hydroxybenzoate, for a cosmetic or dermatological use, without recrystallization of the said alkyl para-hydroxybenzoates, or loss of stability of the composition containing them, and thus to obtain a cosmetically acceptable composition. This use makes it possible in particular to dispense with the use of alcohol, or alternatively to considerably limit the required amount thereof, while at the same time having identical or increased antifungal power.

The use of the lipophilic amino acid derivatives according to the present invention also has an additional advantage when the parabens are introduced into compositions containing a dispersion of solid particles. Specifically, the Applicant has found that the lipophilic amino acid derivatives prevent the adsorption of the alkyl para-hydroxybenzoates onto the surface of the solid particles, the consequence of this phenomenon being a further reduction in the antifungal effect of these parabens.

A subject of the present invention is thus also a composition comprising, in a physiologically acceptable medium, at least one alkyl para-hydroxybenzoate, the alkyl group containing from 1 to 6 carbon atoms, at least one lipophilic amino acid derivative, and at least one dispersion of solid particles.

The alkyl para-hydroxybenzoates used in the compositions according to the invention are advantageously chosen from methyl, propyl and butyl para-hydroxybenzoate, and mixtures thereof.

The lipophilic amino acid derivative is preferably an ester chosen from the amino acid esters of formula (I):
R′₁(CO)N(R′₂)CH(R′₃)(CH₂)ₙ(CO)OR′₄ (I)
in which:
n is an integer equal to 0, 1 or 2,

R′₁ represents a linear or branched C₅ to C₂₁ alkyl or alkenyl radical,

R′₂ represents a hydrogen atom or a C₁ to C₃ alkyl group,

R′₃ represents a radical chosen from the group formed by a hydrogen atom, a methyl group, an ethyl group and a linear or branched C₃ or C₄ alkyl radical,

R′₄ represents a linear or branched C₁ to C₁₀ alkyl radical, a linear or branched C₂ to C₁₀ alkenyl radical or a sterol residue.

These amino acid esters and the process for synthesizing them are described in patent applications EP 1 044 676 and EP 0 928 608 from the company Ajinomoto Co.

In the amino acid esters of formula (I), the group R′₁(CO)—is preferably an acyl group of an acid preferably chosen from the group formed by capric acid, lauric acid, myristic acid, palmitic acid, stearic acid, behenic acid, linoleic acid, linolenic acid, oleic acid, isostearic acid and 2-ethylhexanoic acid, coconut oil fatty acids and palm kernel oil fatty acids. These fatty acids may also contain a hydroxyl group. Even more preferably, the fatty acid will be lauric acid.

The portion —N(R′₂)CH(R′₃)(CH₂)ₙ(CO)— of the amino acid ester is preferably chosen from the following amino acids: glycine, alanine, valine, leucine, isoleucine, serine, threonine, proline, hydroxyproline, β-alanine, aminobutyric acid, aminocaproic acid, sarcosine and N-methyl-β-alanine.

Even more preferably, the amino acid will be sarcosine.

The portion of the amino acid esters corresponding to the group OR′₄ may be obtained from alcohols chosen from the group formed by methanol, ethanol, propanol, isopropanol, butanol, tert-butanol, isobutanol, 3-methyl-1-butanol, 2-methyl-1-butanol, fusel oil, pentanol, hexanol, cyclohexanol, octanol, 2-ethylhexanol, decanol, lauryl alcohol, myristyl alcohol, cetyl alcohol, cetostearyl alcohol, stearyl alcohol, oleyl alcohol, behenyl alcohol, jojoba alcohol, 2-hexadecyl alcohol, 2-octyldodecanol and isostearyl alcohol.

These amino acid esters may be obtained in particular from natural sources of amino acids. In this case, the amino acids are derived from the hydrolysis of natural proteins from plants (oat, wheat, soybean, palm or coconut) and, in this case, necessarily lead to amino acid mixtures that must then be esterified and then N-acylated. The preparation of such amino acids is more particularly described in patent application FR 2 796 550, which is incorporated herein by reference.

The amino acid ester that is more particularly preferred for use in the present invention is isopropyl N-lauroylsarcosinate of formula:
CH₃—(CH₂)₁₀CO—N(CH₃)—CH₂—COO—CH(CH₃)₂.

An example that may be mentioned is Eldew SL-205® sold by the company Ajinomoto.

According to the present invention, the expression “dispersion of solid particles” means any mineral and/or organic solid particle that is insoluble in the medium in which it is dispersed. These particles may be of variable shape; they may in particular be spherical, cylindrical or platelet-shaped. They may also be hollow or solid.

For the purposes of the invention, the term “solid” particles means particles that are solid at room, temperature and atmospheric pressure and that have a melting point of greater than 30° C. The solid/liquid change of state may be reversible.

These solid particles may be solid particles comprising or formed from a crystalline or semi-crystalline material, which is solid at room temperature, mineral and/or organic fibres, of synthetic and/or natural origin, wax microdispersions, silicone resins, silicone elastomers, polyamide particles, microspheres based on acrylic copolymer, expanded powders or silicone resin microbeads.

These particles may especially be pigments or fillers.

An example of solid particles for the purposes of the present invention is represented by solid particles comprising (in particular formed from) a crystalline or semi-crystalline material that is solid at room temperature (25° C.).with a first-order phase transition, of melting or of combustion, of greater than 100° C., preferably greater than 120° C. and better still greater than 150° C.

The melting point or combustion temperature may be measured according to ASTM standard E794-98.

For the purposes of the invention, the term “semi-crystalline material” means a material, especially a polymer, comprising a crystallizable portion and an amorphous portion with a temperature of first-order reversible change of phase, in particular of melting (solid-liquid transition).

Advantageously, the crystalline or semi-crystalline material has a Vickers hardness of greater than or equal to 10, especially ranging from 10 to 7500, preferably greater than or equal to 200, especially ranging from 200 to 7500 and better still greater than or equal to 400, especially ranging from 400 to 7500.

The Vickers hardness (VH) is determined by applying to the material a penetrometer in the form of a square-based pyramid, using a load P. The mean size of a diagonal of the square imprint obtained with the penetrometer is then measured.

The Vickers hardness (VH) is then calculated by means of the relationship:

$$VH = \frac{1854.4 \times P}{{d}^{2}} \begin{matrix}d = mean diameter in μm \\ P = applied load in g\end{matrix}$$

The Vickers hardness may be measured using the M 400 g 2 microdurometer from the company Leco.

The material of the solid particles defined above may be a mineral material, which may be chosen from silica, glass, diamond, copper, boron nitride, ceramics, micas, metal oxides, especially iron oxides such as black iron oxide, red iron oxide or yellow iron oxide, titanium oxides and alumina, and mixtures thereof.

In one advantageous aspect of the invention, the particles contained in the compositions are less than or equal to 20 μm in size. In this case, they will be termed “microparticles”.

Among the solid microparticles that are suitable for implementing the present invention, mention may be made especially of mineral and/or organic fibres, of synthetic and/or natural origin, and also wax microdispersions.

The fibres are preferably chosen from silk fibres, cotton fibres, wool fibres, flax fibres, cellulose fibres extracted especially from wood, from vegetables or from algae, polyamide fibres (Nylon®, especially under the names nylon 6=polyamide 6; nylon 6,6=polyamide 6,6; nylon 12=polyamide 12), rayon fibres, viscose fibres, acetate fibres, especially rayon acetate, cellulose acetate or silk acetate fibres, poly-p-phenyleneterephthamide fibres, especially Kevlar® fibres, acrylic fibres, especially polymethyl methacrylate or poly(2-hydroxyethyl methacrylate) fibres, polyolefin fibres, especially polyethylene fibres or polypropylene fibres, glass fibres, silica fibres, aramid fibres, carbon fibres, especially in graphite form, Teflon® fibres, insoluble collagen fibres, polyester fibres, polyvinyl chloride fibres, polyvinylidene chloride fibres, polyvinyl alcohol fibres, polyacrylonitrile fibres, chitosan fibres, polyurethane fibres, polyethylene phthalate fibres, fibres formed from a blend of polymers such as those mentioned above, for instance polyamide/polyester fibres, and mixtures of these fibres.

The fibres used to implement the present invention preferably have a diameter of between 5 μm and 50 μm and a length of between 20 μm and 1000 μm.

In one particularly advantageous aspect of the present invention, the fibres are chosen from polyamide (Nylon®) fibres, in particular polyamide 6 (Nylon 6), polyamide 6,6 (Nylon 6,6) and polyamide 12 (Nylon 12), and rayon fibres.

For the purposes of the present invention, a wax microdispersion is a dispersion of wax particles, in which the size of the said wax particles is less than or equal to about 1 μm.

In the present patent application, a wax is a lipophilic compound that is solid at room temperature (25° C.), with a solid/liquid reversible change of state, having a melting point of greater than or equal to 30° C., which may be up to 120° C. By bringing the wax to the liquid form (melting), it is possible to make it miscible with oils and to form a microscopically uniform mixture, but on cooling the mixture to room temperature, recrystallization of the wax in the oils of the mixture is obtained.

The melting point of the wax may be measured using a differential scanning calorimeter (D.S.C.), for example the calorimeter sold under the name DSC 30 by the company Mettler. A sample of 15 mg of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

Wax microdispersions are stable dispersions of colloidal particles of wax, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of some of the water, followed by gradual addition of hot water with stirring. The intermediate formation of an emulsion of the water-in-oil type is observed, followed by a phase inversion with final production of an oil-in-water type microemulsion. Upon cooling, a stable microdispersion of solid colloidal wax particles is obtained.

The wax microdispersion may also be obtained by agitating the mixture of wax, surfactant and water with an agitation means, such as ultrasound, a high-pressure homogenizer or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. They may, however, comprise a minor proportion of oily and/or pasty fatty additives, a surfactant and/or a common liposoluble additive/active agent.

The waxes that may be used in the microdispersions according to the invention are chosen from waxes that are solid and rigid at room temperature, of animal, plant, mineral or synthetic origin, and mixtures thereof. The waxes may have a melting point ranging from 30° C. to 120° C. approximately, and better still from 45° C. to 120° C. The wax may also have a hardness ranging from 0.05 MPa to 15 MPa and preferably ranging from 3 MPa to 15 MPa. The hardness is determined by measuring the compression force, measured at 20° C. using a texturometer sold under the name TA-XT2i by the company Rheo, equipped with a stainless-steel cylinder 2 mm in diameter travelling at a measuring speed of 0.1 mm/s, and penetrating into the wax to a penetration depth of 0.3 mm. To perform the hardness measurement, the wax is melted at a temperature equal to the melting point of the wax +20° C. The molten wax is poured into a container 30 mm in diameter and 20 mm deep. The wax is recrystallized at room temperature (25° C.) for 24 hours and is then stored for at least 1 hour at 20° C. before performing the hardness measurement. The hardness value is the measured compression force divided by the area of the texturometer cylinder in contact with the wax.

Mention may also be made of hydrocarbon-based waxes, for instance beeswax, lanolin wax and Chinese insect waxes; rice wax, carnauba wax, candelilla wax, ouricury wax, cork fibre wax, sugar cane wax, Japan wax and sumach wax; montan wax, and waxy copolymers, and also esters thereof.

Mention may also be made of the waxes obtained by catalytic hydrogenation of animal or plant oils containing linear or branched C8-C32 fatty chains.

Among these, mention may be made especially of hydrogenated jojoba oil, hydrogenated sunflower oil, hydrogenated castor oil, hydrogenated coconut oil and hydrogenated lanolin oil.

Mention may also be made of silicone waxes, fluoro waxes, microcrystalline waxes, ceresin or ozokerite, and synthetic waxes, for instance polyethylene waxes and Fischer-Tropsch waxes.

Carnauba wax, beeswax or candelilla wax is preferably used.

It is also possible to use commercial mixtures of self-emulsifying waxes containing a wax and surfactants. These commercial mixtures allow wax microdispersions to be prepared by simple addition of water.

Other solid particles or microparticles that are suitable for use in the present invention may be chosen from silicon resins such as trifluoromethyl-C1-4-alkyl dimethicone and trifluoropropyl dimethicone; silicone elastomers, for instance the products sold under the name “KSG” by the company Shin-Etsu, under the names “Trefil”, “BY299” and “EPSX” by the company Dow Corning or under the name “Gransil” by the company Grant Industries, polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres, and especially the microspheres sold under the name Expancel by the company Kemanord Plast or under the name Micropearl F 80 ED by the company Matsumoto; silicon resin microbeads such as those sold under the name Tospearl by the company Toshiba Silicone; and mixtures thereof.

The concentration of alkyl para-hydroxybenzoate in the composition according to the present invention is between 0.001% and 80%, preferably between 0.01% and 60%, particularly between 0.01% and 10% and even more preferably between 0.05% and 1% by weight relative to the total weight of the composition.

The amount of amino acid esters will depend on the amount of alkyl para-hydroxybenzoate to be dissolved, and may be between 0.01% and 90% by weight, preferably between 0.1% and 30% and more particularly between 0.1% and 10% by weight relative to the total weight of the composition.

When the composition according to the present invention contains solid particles, they represent between 0.05% and 20% and preferably between 0.1% and 10% by weight relative to the total weight of the composition.

The composition according to the invention may be used as a cosmetic composition, in particular to care for the skin and/or mucous membranes, or as a makeup composition, for instance a mascara, or alternatively a composition for treating keratin fibres, especially the eyelashes.

The composition according to the present invention may also be used for the manufacture of a dermatological preparation.

The compositions used according to the invention are intended for topical application to the skin and/or its integuments and thus contain a physiologically acceptable medium, i.e. a medium that is compatible with cutaneous tissues such as the skin, the scalp, the eyelashes, the eyebrows, the hair, the nails and/or mucous membranes. This physiologically acceptable medium may consist more particularly of water and optionally of a physiologically acceptable organic solvent chosen, for example, from lower alcohols containing from 1 to 8 carbon atoms and in particular from 1 to 6 carbon atoms, for instance ethanol, isopropanol, propanol or butanol; polyethylene glycols containing from 6 to 80 ethylene oxide units; polyols, for instance-propylene glycol, isoprene glycol, butylene glycol, glycerol, sorbitol, dipropylene glycol, pentylene glycol and hexylene glycol.

The compositions according to the invention may be in any presentation form conventionally used for topical application and especially in the form of aqueous or aqueous-alcoholic solutions, oil-in-water (O/W) emulsions or water-in-oil (W/O) emulsions or multiple emulsions (triple emulsion: W/O/W or O/W/O), aqueous gels, or dispersions of an oily phase in an aqueous phase using spherules, these spherules possibly being polymer nanoparticles such as nanospheres and nanocapsules, or lipid vesicles of ionic and/or nonionic type (liposomes, niosomes or oleosomes). These compositions are prepared according to the usual methods.

In addition, the compositions used according to the invention may be more or less fluid and may have the appearance of a white or coloured cream, an ointment, a milk, a lotion, a serum, a paste, a mousse or a two-phase solution. They may optionally be applied to the skin in the form of an aerosol. They may also be in solid form, for example in the form of a stick.

They may also be in anhydrous form, in particular in the form of an anhydrous stick.

The composition used according to the invention may also contain fatty substances and/or oils.

As oils which can be used in the composition of the invention, mention may be made for example of:

hydrocarbon-based oils of animal origin, such as perhydrosqualene;

hydrocarbon-based oils of plant origin, such as liquid triglycerides of fatty acids containing from 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter oil;

synthetic esters and ethers, in particular of fatty acids, such as the oils of formulae R¹COOR² and R¹OR² in which R¹ represents a fatty acid residue containing from 8 to 29 carbon atoms and R2 represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, petroleum jelly, polydecenes, hydrogenated polyisobutene such as parleam oil;

fatty alcohols containing from 8 to 26 carbon atoms, such as cetyl alcohol, stearyl alcohol, and the mixture of cetyl alcohol and of stearyl alcohol (cetylstearyl alcohol), Guerbet alcohols such as octyldodecanol and 2-hexyldecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2-295912;

silicone oils such as volatile or non-volatile polymethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendent or at the end of a silicone chain, these groups containing from 2 to 24 carbon atoms; phenylsilicones such as phenyl trimethicones, phenyl dimethicones, phenyltrimethyl-siloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

The term “hydrocarbon-based oil” in the list of the abovementioned oils embraces any oil comprising predominantly carbon and hydrogen atoms, and optionally ester, ether, fluoro, carboxylic acid and/or alcohol groups.

The other fatty substances which may be present in the oily phase are, for example, fatty acids containing from 8 to 30 carbon atoms, for instance stearic acid, lauric acid, palmitic acid and oleic acid.

These fatty substances may be chosen in a varied manner by a person skilled in the art in order to prepare a composition having the desired properties, for example consistency or texture properties.

According to one particular embodiment of the invention, the composition according to the invention is a water-in-oil (W/O) or oil-in-water (O/W) emulsion. The proportion of oily phase of the emulsion may range from 5% to 80% by weight and preferably from 5% to 50% by weight relative to the total weight of the composition.

The emulsions generally contain at least one emulsifier chosen from amphoteric, anionic, cationic and nonionic emulsifiers, used alone or as a mixture, and optionally a co-emulsifier. The emulsifiers are chosen in an appropriate manner depending on the emulsion to be obtained (W/O or O/W). The emulsifier and, the co-emulsifier are generally present in the composition in a proportion ranging from 0.3% to 30% by weight and preferably from 0.5% to 20% by weight relative to the total weight of the composition.

Examples of emulsifiers that may be mentioned for the W/O emulsions include dimethicone copolyols such as the mixture of cyclomethicone and of dimethicone copolyol, sold under the name “DC 5225 C” by the company Dow Corning, and alkyldimethicone copolyols, such as the laurylmethicone copolyol sold under the name “Dow Corning 5200 Formulation Aid” by the company Dow Corning, and the cetyldimethicone copolyol sold under the name Abil EM 90® by the company Goldschmidt. Surfactants of W/O emulsions that may also be used include a crosslinked elastomeric solid organopolysiloxane comprising at least one oxyalkylenated group, such as those obtained according to the procedure of Examples 3, 4 and 8 of document U.S. Pat. No. 5,412,004 and the examples of document U.S. Pat. No. 5,811,487, especially the product of Example 3 (synthesis example) of patent U.S. Pat. No. 5 412 004, such as the product sold under the reference KSG 21 by the company Shin Etsu.

For the O/W emulsions, examples of emulsifiers that may be mentioned include nonionic emulsifiers such as oxyalkylenated fatty acid esters of sorbitan and of glycerol; oxyalkylenated (oxyethylenated and/or bxypropylenated) fatty alcohol ethers; sugar esters, for instance sucrose stearate; and mixtures thereof.

In a known manner, the cosmetic or dermatological composition of the invention may also contain adjuvants that are common in cosmetics or dermatology, such as active agents, antioxidants, hydrophilic or lipophilic gelling agents, solvents, fragrances, UV-screening agents, odour absorbers, dyestuffs, plant extracts and salts. The amounts of these various adjuvants are those conventionally used in the field under consideration, for example from 0.01% to 20% relative to the total weight of the composition.

Depending on their nature, these adjuvants may be introduced into the fatty phase, into the aqueous phase and/or into lipid spherules.

According to one preferred embodiment, the compositions used in accordance with the invention may also comprise at least one UVA-active and/or UVB-active organic photoprotective agent and/or at least one mineral photoprotective agent (absorbers), which are water-soluble or liposoluble, or even insoluble in the cosmetic solvents commonly used.

The organic photoprotective agents are chosen especially from anthranilates; cinnamic derivatives; dibenzoylmethane derivatives; salicylic derivatives, camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzo-triazole derivatives.; benzalmalonate derivatives; benzimidazole derivatives; imidazolines; bis-benzazolyl derivatives as described in patents EP 669 323 and U.S. Pat. No. 2 463 264; p-aminobenzoic acid (PABA) derivatives; methylenebis(hydroxyphenylbenzotriazole) derivatives as described in patent applications U.S. Pat. No. 5 237 071, U.S. Pat. No. 5 166 355, GB 2 303 549, DE 197 26 184 and EP 893 119; screening polymers and screening silicones such as those described especially in patent application WO 93/04665; dimers derived from α-alkylstyrene, such as those described in patent application DE 198 55 649; and mixtures thereof.

The photoprotective agents are generally present in the compositions according to the invention in proportions ranging from 0.1% to 20% by weight relative to the total weight of the composition, and preferably ranging from 0.2% to 15% by weight relative to the total weight of the composition.

The compositions according to the invention may optionally contain one or more thickening compounds, in concentrations preferably ranging from 0.05% to 2% by weight relative to the total weight of the composition.

As examples of thickening compounds that may be used in the composition of the invention, mention may be made of:

polysaccharide biopolymers, for instance xanthan gum, guar gum, alginates and modified celluloses;

synthetic polymers, such as polyacrylics, for instance Carbopol 980 sold by the company Goodrich, and acrylate/acrylonitrile copolymers such as Hypan SS201 sold by the company Kingston;

mineral compounds such as modified or unmodified smectites and hectorites, such as the Bentone products sold by the company Rheox, the Laponite products sold by the company Southern Clay Products, or the product Veegum HS sold by the company R. T. Vanderbilt;.

and mixtures thereof.

The present invention also relates to a process for dissolving at least one alkyl para-hydroxybenzoate, the alkyl group containing from 1 to 6 carbon atoms, comprising the step consisting in mixing it with at least one amino acid ester of formula (I):
R′₁(CO)N(R′₂)CH(R′₃)(CH₂)ₙ(CO)OR′₄ (I)
in which:
n is an integer equal to 0, 1 or 2,
R′₁ represents a linear or branched C₅ to C₂, alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical,
R′₄ represents a linear or branched C₁ to C₁₀ alkyl radical, a linear or branched C₂ to C₁₀ alkenyl radical, or a sterol residue.

According to one preferred embodiment of the invention, the alkyl para-hydroxybenzoate/amino acid ester ratio is between 0.001/99.999 and 70/30 and better still between 20/80 and 60/40.

A subject of the present invention is also the use of at least one amino acid ester of formula (I):
R′₁(CO)N(R′₂)CH(R′₃)(CH₂)ₙ(CO) OR′₄ (I)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical,
R′₄ represents a linear or branched C₁ to C₁₀ alkyl radical., a linear or branched C₂ to C₁₀ alkenyl radical, or a sterol residue,
to prevent the adsorption of at least one alkyl para-hydroxybenzoate onto solid particles.

The solid particles are chosen in particular from solid microparticles and preferably mineral and/or organic fibres, of synthetic and/or natural origin, and also wax microdispersions, or mixtures thereof.

A subject of the invention is also a cosmetic skincare and/or makeup process, characterized in that it comprises the application to the skin, mucous membranes and/or keratin fibres of a composition according to the invention.

The examples that follow illustrate the invention without limiting its scope. Depending on the case, the compounds are cited as chemical names or as CTFA names (International Cosmetic Ingredient Dictionary and Handbook).

### EXAMPLE 1

### Solubility

Protocol:

The alkyl para-hydroxybenzoates are weighed out and placed in a hermetic pill bottle. The required amount of lipophilic amino acid derivative (solubilizer) is added.

The suspension is brought to 80° C. and stirred. by magnetic stirring for one hour. The dissolution or non-dissolution of the alkyl para-hydroxybenzoate and its change over time are then monitored.

The insolubility of the alkyl para-hydroxybenzoate in the solubilizer is characterized macroscopically by a precipitate or just a cloudy solution, and microscopically by the presence of crystals.

Results:

A test conducted with isopropyl N-lauroyl-sarcosinate as lipophilic amino acid derivative made it possible to dissolve up to 40% by weight of methyl para-hydroxybenzoate (methyl paraben) (the remainder of the solution consisting of the solubilizer).

For comparative purposes, the same amount of methyl paraben in isononyl isononanoate (conventionally used solvent) leads to the formation of insoluble crystals and a paste.

Similarly, a test performed with isopropyl N-lauroylsarcosinate as lipophilic amino acid derivative made it possible to dissolve up to 60% by weight of propyl para-hydroxybenzoate (propyl paraben) (the remainder of the solution consisting of the solubilizer).

For comparative purposes, the same amount of propyl paraben in water (conventionally used solvent) leads to the formation of insoluble crystals and a paste.

### EXAMPLE 2

### Formulation Examples

Several examples were performed in different types of emulsion.

### EXAMPLE A

### Emulsion Based on Sugar Esters

| Phase A: | |
|---|---|
| ammonium polyacryloyldimethyl taurate | 1.5% |
| sodium hyaluronate | 0.1% |
| glycerol | 5% |
| polyamide fibres 6 | 8% |
| water | qs 100% |
| Phase B: | |
| PEG 120 methyl glucose dioleate | 0.5% |
| polysorbate 20 | 0.5% |
| isopropyl N-lauroylsarcosinate | 10% |
| propyl paraben | 0.5% |
| Phase C: | |
| ethanol | 5% |

Procedure:

The aqueous phase A is prepared by mixing together the various constituents and homogenizing. Phase B is prepared by adding the premix of propyl paraben and of solubilizer to the surfactant mixture. Phase A is then added to phase B at a temperature of 75° C. Phase C is then added at a temperature of 25° C. to the mixture obtained.

### EXAMPLE B

### Fluid Emulsion

| Phase A: | |
|---|---|
| carbomer | 0.2% |
| preserving agent | 0.65% |
| acrylates/C₁₀₋₃₀ alkyl acrylate | 0.2% |
| crosspolymer | |
| glycerol | 3% |
| antioxidant | 0.3% |
| xanthan | 0.2% |
| base | 0.2% |
| water | qs 100% |
| Phase B: | |
| cyclohexasiloxane | 5% |
| isopropyl N-lauroylsarcosinate | 1.0% |
| methyl paraben | 2% |
| Phase C: | |
| dimethicone copolyol | 5% |
| Phase D: | |
| polyacrylamide (and) C₁₃-C₁₄ | 0.4% |
| isoparaffin (and) Laureth-7 | |
| Phase E: | |
| ethanol | 5% |

Procedure:

Phase A is prepared by homogenizing the various constituents. The premix of methyl paraben and solubilizer is added to the other constituent of the oily phase B. Phase B is added to phase A at a temperature of 30° C. Next, phases C, D and E are successively added.

### EXAMPLE C

A microdispersion of carnauba wax having the composition below was prepared:

| | | |
|---|---|---|
| carnauba wax | 27 | g |
| polyoxyethylenated (30 EO) glyceryl | 6.75 | g |
| monostearate (Tagat S from Goldschmidt) | | |
| ethanol | 10 | g |
| water | qs 100 | g |

The wax and the surfactant were heated to 90° C., while homogenizing the mixture with moderate stirring. The water heated to 90° C. was then incorporated with continued stirring. The mixture was cooled to room temperature and the ethanol was added to obtain a wax microdispersion with a mean particle diameter of about 170 nm.

### EXAMPLE D

### Care Fluid

| Phase A: | |
|---|---|
| water | 43% |
| glycerol | 3% |
| methyl paraben | 0.4% |
| trisodium EDTA | 0.05% |
| Phase B: | |
| cyclohexasiloxane | 7% |
| glyceryl stearate/PEG-100 | 3.8% |
| stearate/polysorbate 60/cetyl | |
| alcohol/stearic acid | |
| butyl paraben | 0.15% |
| isopropyl N-lauroylsarcosinate | 5.0% |
| fragrance | 0.1% |
| Phase C: | |
| water | 10% |
| ammonium polyacryloyldimethyl- | 1% |
| taurate | |
| Phase D: | |
| water | qs 100% |
| terephthalidenedicamphorsulphonic | 0.7% |
| acid | |
| phenylbenzimidazolesulphonic acid | 2% |
| triethanolamine | qs pH = 6.5 |
| Phase E: | |
| acrylate copolymer | 0.3% |
| Phase F: | |
| microdispersion according to | 17.5% |
| Example C | |

Phase A is heated with stirring to 80° C. until dissolution is complete. Phase B is heated with stirring to 80° C. until a clear phase is obtained, and is then added to phase A with stirring. The mixture is then cooled to 60° C. The-ammonium polyacryloyldimethyl-taurate is swollen in the water at 60° C. for 10 minutes and phase C is added to-the mixture of phases A+B.

Phase D is dissolved with stirring at 50° C. and then added to the mixture of phases A+B+C. The resulting mixture is then cooled to 30° C. Phases E and F are successively introduced at 30° C. The temperature of the mixture is then returned to 20° C.

### EXAMPLE E

### Care Cream:

The composition below is prepared in a manner that is conventional to those skilled in the art.

| | |
|---|---|
| Octyldodecanol | 1% |
| Polysorbate 60 | 0.7% |
| Stearic acid | 0.5% |
| Glyceryl stearate/PEG-100 stearate | 1.6% |
| Isopropyl N-lauroylsarcosinate | 3.0% |
| Disodium EDTA | 0.2% |
| Neutralizers | 0.2% |
| Gelling agents | 2.0% |
| Glycerol | 3.0% |
| Methyl paraben | 0.3% |
| Propyl paraben | 0.2% |
| Butyl paraben | 0.15% |
| 5-n-Octylsalicylic acid | 0.1% |
| Cetyl alcohol | 1.0% |
| Cyclohexasiloxane | 1.0% |
| Ascorbyl glucoside | 0.05% |
| Polyamide 6 fibres* | 8% |
| Water | qs 100% |

*the fibres have a diameter of 10 μm and a length of 300 μm.

1-18. (canceled)

19. A cosmetic composition, comprising:
a physiologically acceptable medium,
at least one alkyl para-hydroxybenzoate, and
at least one lipophilic amino acid derivative,
wherein the alkyl para-hydroxybenzoate has an alkyl group containing from 1 to 6 carbon atoms.

20. The composition according to claim 19, wherein the alkyl para-hydroxybenzoate is at least one selected from the group consisting of methyl para-hydroxybenzoate, propyl para-hydroxybenzoate, and butyl para-hydroxybenzoate.

21. The composition according to claim 19, wherein the lipophilic amino acid derivative is at least one amino acid ester of formula (I):
R′₁(CO)N(R′₂)CH(R′₃)(CH₂)ₙ(CO)OR′₄ (I)
in which:
n is an integer equal to 0, 1 or 2,
R′₁ represents a linear or branched C₅ to C₂, alkyl or alkenyl radical,
R′₂ represents a hydrogen atom or a C₁ to C₃ alkyl group,
R′₃ represents a radical selected from the group consisting of a hydrogen atom, a methyl group, an ethyl group, a linear C₃ alkyl radical, a linear C₄ alkyl radical, a branched C₃ alkyl radical, and a branched C₄ alkyl radical, and
R′₄ represents a linear C₁ to C₁₀ alkyl radical, a branched C₁ to C₁₀ alkyl radical, a linear C₂ to C₁₀ alkenyl radical, a branched C₂ to C₁₀ alkenyl radical or a sterol residue.

22. The composition according to claim 21, wherein the amino acid ester is isopropyl N-lauroylsarcosinate:
CH₃—(CH₂)₁₀CO—N(CH₃)—CH₂—COO—CH(CH₃)₂.

23. The composition according to claim 19, wherein the alkyl para-hydroxybenzoate is present in an amount from 0.001% to 80% by weight relative to the total weight of the composition.

24. The composition according to claim 19, wherein the alkyl para-hydroxybenzoate is present in an amount of from 0.01% to 60% by weight relative to the total weight of the composition.

25. The composition according to claim 19, wherein the alkyl para-hydroxybenzoate is present in an amount of from 0.01% to 10% by weight relative to the total weight of the composition.

26. The composition according to claim 19, wherein the alkyl para-hydroxybenzoate is present in an amount of from 0.05% to 1% by weight relative to the total weight of the composition.

27. The composition according to claim 19, wherein the lipophilic amino acid derivative is present in an amount of from 0.01% to 90% by weight relative to the total weight of the composition.

28. The composition according to claim 19, wherein the lipophilic amino acid derivative is present in an amount of from 0.1% to 30% by weight relative to the total weight of the composition.

29. The composition according to claim 19, wherein the lipophilic amino acid derivative is present in an amount of from 0.1% to 10% by weight relative to the total weight of the composition.

30. The composition according to claim 19, further comprising:
at least one dispersion of solid particles.

31. The composition according to claim 30, wherein the solid particles are microparticles having a size of less than or equal to 20 μm.

32. The composition according to claim 30, wherein the solid particles are at least one selected from the group consisting of synthetic mineral fibers, natural mineral fibers, synthetic organic fibers, natural organic fibers, and wax microdispersions.

33. The composition according to claim 32, comprising fibers selected from the group consisting of silk fibers, cotton fibers, wool fibers, flax fibers, cellulose fibers, polyamide fibers, rayon fibers, viscose fibers, acetate fibers, poly-p-phenyleneterephthalamide fibers, acrylic fibers, polyolefin fibers, glass fibers, silica fibers, aramid fibers, carbon fibers, polytetrafluoroethylene fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, blends thereof, and mixtures thereof.

34. The composition according to claim 32, comprising fibers selected from the group consisting of cellulose fibers extracted from wood, cellulose fibers extracted from vegetables, cellulose fibers extracted from algae, rayon acetate fibers, cellulose acetate fibers, silk acetate fibers, polymethylmethacrylate fibers, poly(2-hydroxyethyl methacrylate) fibers, polyethylene fibers, polypropylene fibers, graphite fibers, polyamide/polyester fibers, blends thereof and mixtures thereof.

35. The composition according to claim 32, comprising fibers selected from the group consisting of polyamide fibers, rayon fibers and mixtures thereof.

36. The composition according to claim 32, comprising at least one wax microdispersion selected from the group consisting of a microdispersion of a hydrocarbon wax, a wax obtained by catalytic hydrogenation of animal or plant oils containing linear or branched C₈-C₃₂ fatty chains, a silicone wax and a fluorowax.

37. The composition according to claim 32, comprising at least one wax microdispersion selected from the group consisting of a microdispersion of beeswax, a microdispersion of lanolin wax, a microdispersion of a Chinese insect wax, a microdispersion of a rice wax, a microdispersion of a carnuba wax, a microdispersion of a candelilla wax, a microdispersion of an ouricury wax, a microdispersion of a cork fiber wax, a microdispersion of a sugar cane wax, a microdispersion of a Japan wax, a microdispersion of a sumach wax, a microdispersion of a montan wax, a microdispersion of one or more waxy copolymers, a hydrogenated jojoba oil, a hydrogenated sunflower oil, a hydrogenated castor oil, a hydrogenated coconut oil, a hydrogenated lanolin oil and esters thereof.

38. The composition according to claim 31, wherein the solid particles are present in an amount of from 0.05% to 20% by weight relative to the total weight of the composition.

39. The composition according to claim 31, wherein the solid particles are present in an amount of from 0.1% to 10% by weight relative to the total weight of the composition.

40. A process for dissolving at least one alkyl para-hydroxybenzoate having an alkyl group containing from 1 to 6 carbon atoms, comprising:
mixing the alkyl para-hydroxybenzoate with at least one amino acid ester of formula (I):
R′₁(CO)N(R′₂)CH(R′₃)(CH₂)ₙ(CO)OR′₄ (I)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical selected from the group consisting of a hydrogen atom, a methyl group, an ethyl group, a linear C₃ alkyl radical, a linear C₄ alkyl radical, a branched C₃ alkyl radical, and a branched C₄ alkyl radical, and
R′₄ represents a linear C₁ to C₁₀ alkyl radical, a branched C₁ to C₁₀ alkyl radical, a linear C₂ to C₁₀ alkenyl radical, a branched C₂ to C₁₀ alkenyl radical or a sterol residue; to dissolve the alkyl para-hydroxybenzoate.

41. The process according to claim 40, wherein the alkyl para-hydroxybenzoate and the amino acid ester are present in an alkyl para-hydroxybenzoate/amino acid ester ratio of between 0.001/99.99 and 70/30.

42. The process according to claim 40, wherein the alkyl para-hydroxybenzoate and the amino acid ester are present in an alkyl para-hydroxybenzoate/amino acid ester ratio of between 20/80 and 60/40.

43. A process, comprising:
treating a mixture comprising one or more solid particles with at least one amino acid ester of formula (I):
R′₁(CO)N(R′₂)CH(R′₃)(CH₂)ₙ(CO)OR′₄ (I)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical selected from the group consisting of a hydrogen atom, a methyl group, an ethyl group, a linear C₃ alkyl radical, a linear C₄ alkyl radical, a branched C₃ alkyl radical, and a branched C₄ alkyl radical, and
R′₄ represents a linear C₁ to C₁₀ alkyl radical, a branched C₁ to C₁₀ alkyl radical, a linear C₂ to C₁₀ alkenyl radical, a branched C₂ to C₁₀ alkenyl radical or a sterol residue; to prevent the adsorption of at least one alkyl para-hydroxybenzoate onto the solid particles.

44. The process of claim 43, wherein the alkyl para-hydroxybenzoate has an alkyl group having from 1 to 6 carbon atoms.

45. The process of claim 43, wherein the solid particles are present in the form of a dispersion in a physiologically acceptable medium.

46. The process of claim 43, wherein the treating includes mixing the amino acid ester and the alkyl para-hydroxybenzoate in the presence of the solid particles.

47. The process of claim 46, wherein the amino acid ester and the para-hydroxybenzoate are mixed with a dispersion of the solid particles in a physiologically acceptable medium.

48. The process according to claim 43, wherein the solid particles are at least one selected from the group consisting of synthetic mineral fibers, natural mineral fibers, synthetic organic fibers, natural organic fibers, and wax microdispersions.

49. A cosmetic process, comprising:
applying the cosmetic composition of claim 19 to at least one of the skin, mucous membranes and keratin fibers of a human.
